(19) United States
(12) Patent Application Publication
(10) Pub. No.: US 2021/0290493 A1
(43) Pub. Date: Sep. 23, 2021

(54) GUIDE WIRE FOR USE IN TUBULAR MEDICAL PROBES, IN PARTICULAR FOR NUTRITIONAL THERAPY

(71) Applicant: Haeberle Laser- und Feinwerktechnik GmbH & Co. KG, Schramberg (DE)

(72) Inventor: Thomas FRIETSCH, Dunningen (DE)

(73) Assignee: Haeberle Laser- und Feinwerktechnik GmbH & Co. KG, Schramberg (DE)

(21) Appl. No.: 16/319,609

(22) PCT Filed: Aug. 2, 2017
(86) PCT No.: PCT/EP2017/000937

§ 371 Date: Jan. 22, 2019

(30) Foreign Application Priority Data
Aug. 12, 2016 (DE) 102016009871.6

(51) Int. Cl. A61J 15/00; A61M 25/09

(52) U.S. Cl. CPC A61J 15/0007; A61M 25/09

(57) ABSTRACT

A guide wire is provided with radial play for use as reinforcement in a soft probe tube, and has behind an insertion aid first a more flexible portion, before it is adjoined by the conventional guide wire. In that portion, the insertion aid and the guide wire grip coaxially into oppositely located ends of a tubular annular spring, in particular a flat wire coil spring wound on a block.

The invention relates to a guide wire, in particular for use in the interior of a tubular nutritional probe, as mentioned in DE 10 2004 023 078 B3 (paragraph [0005]).

Conventional tubular transnasal stomach probes for supplying food, fluids and medicaments consist of pliable material to prevent injuries to the esophagus during insertion of the probe tube. Advancing it from the nose therefore requires a reinforcement to prevent the tube from bending or even buckling in the course of its advancement. According to DE 297 18 991 U1, for this a wire is pushed in or formed within the wall of the probe tube in an axially parallel manner.

It is standard medical practice to arrange in the interior space of a tube a guide wire which, during the insertion and for navigation of the probe, is made to follow the advancement of the probe tube by being pushed thereafter in steps forward and back as a temporary internal reinforcement. The advancing, withdrawing, and possibly turning and further advancing, of the guide wire have the effect that the distal end of the probe is placed in its final position in the stomach or in the intestine after negotiating branches such as are encountered for instance in the nasal passage or in the stomach. The guide wire can subsequently be withdrawn in order to open up the entire cross section of the tube, in particular for feeding in food or medicaments.

The guide wire is usually a solid, acid-resistant thin stainless steel spring wire, a stranded wire or a continuous hollow-cylindrical round-wire helical spring, in each case with a spherical insertion aid situated at the distal end. However, the comparatively low flexibility of the guide wire entails the risk that, in the course of its advancement, it punctures the probe tube at deflections with a small radius or even buckling points. Then, the attempt to make further advancements, or even to withdraw the probe, leads to injuries, for instance of a patient's esophagus. In the case of a guide wire formed as a continuous round-wire helical spring, it is detrimental that an introduction of tensile force leads to an extension of the spring that is no longer sufficiently reversed if the probe is already far advanced, and thus hinders or even prevents the final positioning of the probe.

In recognition of such circumstances, the present invention is based on the technical problem of providing a guide wire which on the one hand minimizes the risk of puncturing the probe tube and on the other hand responds to the latest deformation, even if of a small bending radius or long extent, by automatically aligning itself again to a certain degree for as far as possible trouble-free further navigation.

This is not achievable in the way known from WO 2011/038522 A1 by creating a hollow guide wire which, for the radial passing through of fluid to be fed in or removed, has proximally ahead of a hemispherical insertion aid a flexible round-wire helix, wound with axial spacing, distally ahead of the hollow rest of the guide wire. For a navigation of the guide wire, it is required that axially parallel through its cavity a core wire additionally extends through the wire helix into the insertion aid. This insertion aid, a hemisphere of plastic, is connected by pressure welding to the wire helix ahead of its distal end.

The requirements according to DE 697 09 997 T2 are not satisfied either. There, a guide wire of a similar type is surrounded along the distal region of the tapered end portion by a fine wire coil, in particular of platinum, wound at great axial winding intervals, in order to increase the buckling resistance here without significant loss of flexibility. The actual guide wire, which here is tapered and enclosed in fabric, extends axially through the wire coil until it butts against a hemispherical insertion aid, which is bonded in a butting manner ahead of the distal ends in line with one another of the wire coil and the guide wire.

On the other hand, that problem is solved according to the invention by the features specified in the independent patent claim. Accordingly, between the conventional guide wire described at the beginning and its distal insertion aid, a transitional region is inserted in the form of a more flexible portion in comparison with the guide wire, a transitional region which is designed as an annular tube spring with or without prestress, preferably as a flat-wire helical spring wound on a block.

This measure promotes during advancement a deflection of that portion ahead of the guide wire when the latest run of the sensor tube in relation to the guide wire leads to a radial or transverse pressure being exerted by the tube inner wall on the insertion aid. The reduced stiffness of this portion reduces the abutting force of the insertion aid against the supporting point in the interior of the tube. With further advancement, the insertion aid can move away in the direction of the middle of the tube; this virtually eliminates the risk of the tube wall being punctured here from the inside.

In a development of the solution according to the invention, it is taken into consideration that the distal end region of the guide wire cannot however move away any longer if, when there is a buckled tube, its remaining inner cross section is smaller than the transverse dimension of the insertion aid. Then, further repeated exertion of longitudinal pressure on the guide wire leads at most to a slight elastic stretching of the probe tube; and in any event the more flexible portion ahead of the then-following rest of the guide wire buckles elastically in a radially arcuate manner within the cross section available in the interior tube space behind the buckling point, to finally also deform the tube correspondingly. As a result of the force vector decomposition occurring as a result when the insertion aid is blocked, a large part of the forces occurring is dissipated via the wall of the tube; and only such a small part of the longitudinal pressure exerted on the guide wire for navigation acts on the insertion aid that there is virtually no longer the risk of the buckling point perforating.

The portion of increased flexibility inserted according to the invention distally ahead of the rest of the guide wire is realized by inserting a helical spring with a reduced spring constant in comparison with that of the proximally adjoining conventional guide wire in the form of a steel spring wire or a stranded wire. The distal end of the guide wire, on the one hand flexible and on the other hand resistant to shearing, helps the insertion of the probe.

The helical spring, wound on a block and inserted distally into the guide wire as a more flexible portion, generally has the advantage on the basis of the system used of being able to transfer great axial forces. Furthermore, under transverse loading (for instance as a result of the probe being supported laterally against a region in the human body), it does not undergo any permanent deformation, even after a run with a small bending radius, but instead returns from the loading-induced form of running in an angled-away manner back into the original linear extent after the exposure to transverse force is ended.

In the case of the mechanically weakened portion proximally adjoining behind the insertion aid in the form of a more flexible helical spring, it is preferably wound as a single layer of flat wire, in particular of rolled flat wire. Such a helical spring has the additional benefit of being able to absorb greater tensile forces before the occurrence of permanent deformation in comparison with the round-wire spring of a comparable wire cross section. That is useful when a not yet finally positioned probe has become caught in the body, and therefore has to be carefully recovered by the guide wire by using increased tensile force. The permissible tensile stress can be increased if the flat-wire helical spring is wound under prestress with its turns lying axially against one another. On the other hand, a flat-wire helical spring wound with its turns axially spaced would have only a small spring constant.

Additional developments and modifications of the solution according to the invention are provided by the further claims and, while taking their advantages into consideration, from the following description of preferred embodiments of the invention, which are outlined in the drawing in a greatly enlarged form approximately to scale. Depicted in a broken-away representation in the drawing, in each case with a restriction to what is functionally essential:

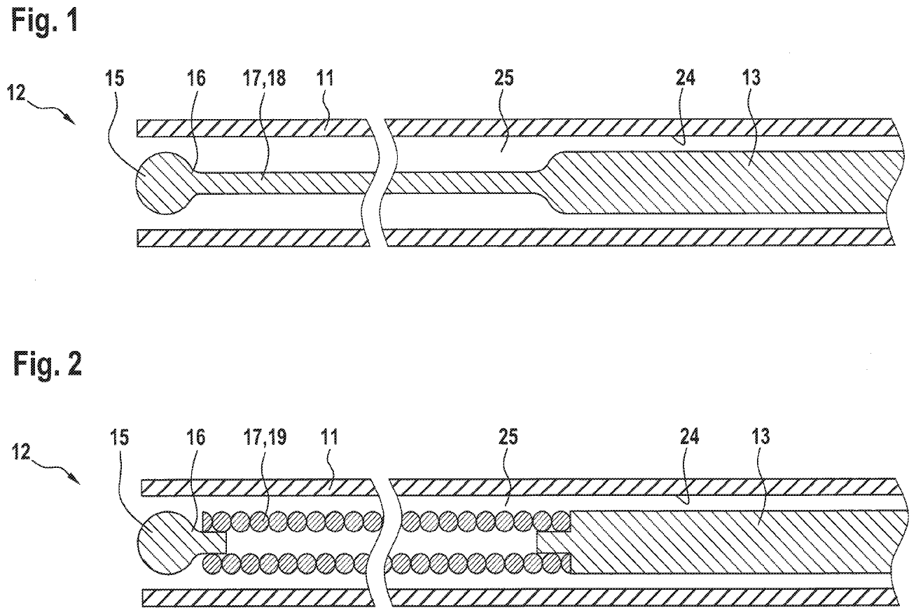

FIG. 1 shows in the probe tube a more flexible since narrowed portion of a solid or stranded guide wire as a transition from an insertion aid to the conventional rest of the guide wire according to the prior art and

FIG. 2 shows in the probe tube a helical spring as such a more flexible portion according to the invention;

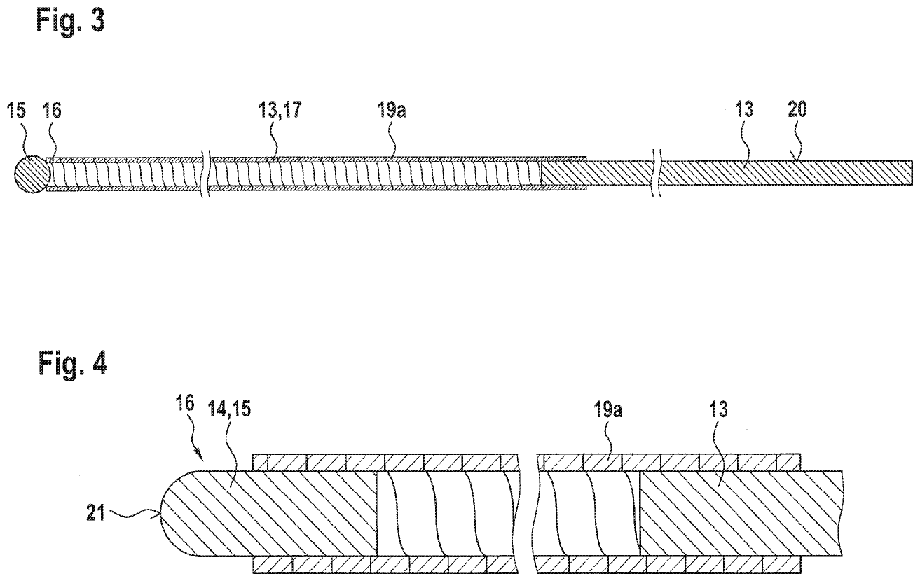

FIG. 3 shows a flat-wire helical spring between the distal insertion aid and the proximal connection to the rest of the guide wire,

FIG. 4 shows as an insertion aid a short round wire that is rounded off at the end in a flat-wire helical spring;

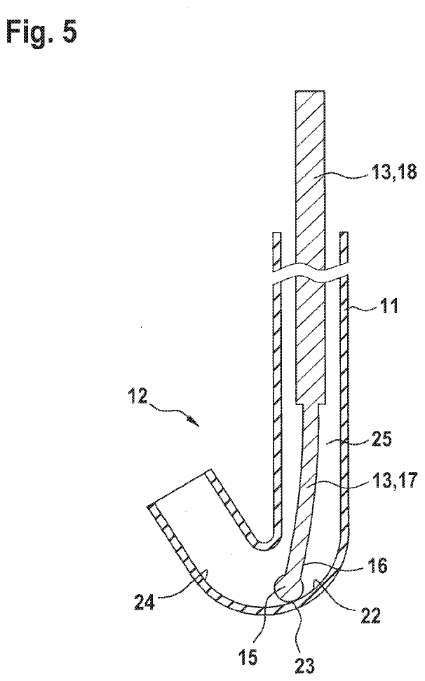

FIG. 5 shows a distally weakened guide wire supported against a deflection of the probe tube at risk of perforation and

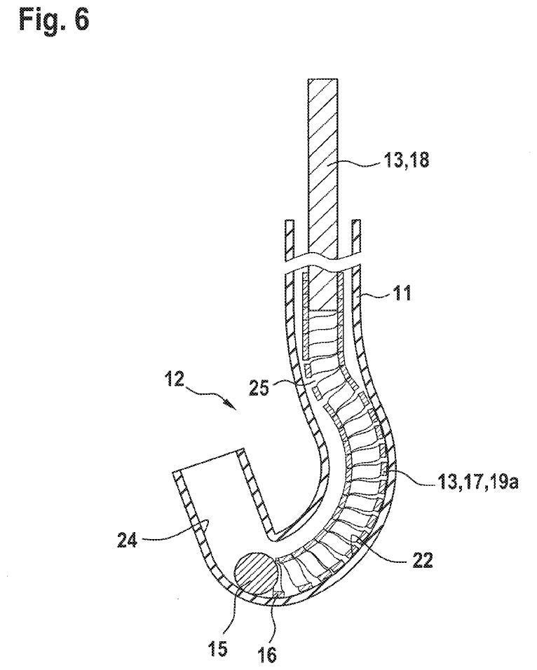

FIG. 6 shows the action of a flat-wire helical spring in a situation approximately according to FIG. 5.

The pliant tube 11, for instance of a nasogastric probe 12, undergoes over its length of typically 50 to 150 cm during the insertion into the patient an internal reinforcement, while having sufficient radial play with respect to the tube inner wall 24, by means of a stranded wire or by means of a solid spring wire of acid-resistant stainless steel as a guide wire 13 of a diameter in the range of typically about 0.75 mm to 1 mm. The distal end 16 of the guide wire 13 is provided with an insertion aid 15 that is rounded off at the end. Its diameter may be much greater than that of the guide wire 13, but it is less than the inside diameter of the tube 11; so that—after the navigation of the probe 12—gastric acid can be drawn off through the tube 11 without any complication, in order to verify the correct position of the probe. For commercially available tubes 11, with an inside diameter of 2 mm to 3 mm, the diameter of a spherical insertion aid 15 at the end is about 1.3 mm. Proximally following the insertion aid 15 there is first a portion 17 of reduced flexural stiffness of the guide wire 13, before it is proximally adjoined by the stiffer conventional rest of the guide wire 13.

This more flexible portion 17, typically about 5 cm long, between the insertion aid 15 and the rest of the guide wire 13 is provided with a single or multiple wire 18 that is thinner by about 20% to 50% in comparison with the thickness of the rest of the guide wire 13.

According to the prior art, the wire 18 may be for example an unspliced central part of the strands of the guide wire 13, or a solid stainless steel spring wire welded in between the insertion aid 15 and the rest of the guide wire 13; according to the invention, however, it is a helical spring 19 according to FIG. 2, in particular a flat-wire helical spring 19a according to FIG. 3.

The free end of such a spring, also known as an annular tube spring, is preferably ground transversely to the longitudinal axis, so that the convex insertion aid 15 to be welded on in front in the form of a prefabricated steel sphere engages in a self-centering manner in the distal end turn of this helical spring 19 or 19a.

In the case of the exemplary embodiment according to FIG. 3/FIG. 4, the portion 17 in the form of such a flat-wire helical spring 19a is fixed in a frictionally engaging or material-bonding manner on the outer lateral surface 20 of the rest of the guide wire 13 by its greater spring stiffness in comparison with the portion 17. For this, the rest of the guide wire 13 engages in the way shown with its distal end coaxially in the helical spring 19 or 19a by a few turns. Axially opposite, the outside diameter of the helical spring 19/19a in the case that is represented by way of example in FIG. 3 of a spherical insertion aid 15 fastened distally in front is slightly overlapped radially by it.

According to FIG. 3/FIG. 4, the helical spring 19a is wound from (rolled) flat wire, preferably under prestress between the successive turns on a block. Then, this portion 17 cannot move away as easily under axial compressive loading. On the other hand, this produces the advantage that higher tensile forces can be introduced under tensile loading for withdrawing the guide wire 13 from the positioned tube 11 without any complication, without permanently deforming the flat-wire helical spring 19a thereby.

The welding operation between the small dimensions for mounting an insertion aid 15 as shown in FIG. 3 is critical in process engineering terms. To be preferred, therefore, is to distally round a wire-shaped flexible portion 17 according to FIG. 1; or to make a short linear portion of wire 14 (FIG. 4) of the order of magnitude of 3-4 mm in length engage instead of a sphere in the distal end of the helical spring 19 or 19a and fix it coaxially therein in a material-bonding manner, in particular by welding. If this pin-shaped insertion aid 15 is not already supplied in such a rounded-off form, the distally projecting end of the portion of wire 14 can without any problem be given a rounding 21 by laser melting forming after mounting. In any event, with this development according to the invention of the use of a short linear portion of wire 14 according to FIG. 4, there is no need for the requirement of grinding the spring transversely to the longitudinal axis, since a sphere no longer has to be centered on the distal end of the annular tube spring.

If, during its advancement, the probe tube 11 has undergone a deflection 22, for instance according to FIG. 5, then conventionally there is the risk here, at the supporting point 23, of being perforated by the insertion aid 15 at the end 16 of a relatively stiff, and therefore virtually linearly pushed-after guide wire 13 according to the prior art.

This risk is then averted by the more flexible front portion 17 according to the invention of the guide wire 13; especially if, according to FIG. 6, the insertion aid 15 is followed proximally by the more flexible portion 17 of the guide wire 13 in the form of a flat-wire helical spring 19a. This is so because then, when axial pressure is exerted externally on the guide wire 13, the insertion aid 15 can approximately follow the course of the bend of the probe 12 along the tube inner wall 24. The helical spring 19a is therefore elastically deformed, which brings about a restoring force into the linear series of flat wire turns; with the tendency of straightening out the course of the deflection 22. Also, the relatively more rigid rest of the guide wire 13 following the portion 17 in the bend 22 of the probe exerts a straightening moment on this deflection 22, which to some degree straightens the bending, when it is pushed after the portion 17. In this case, a portion 17 in the form of a helical spring 19 delivers a greater straightening moment than a spring wire or even a stranded wire, which leaves greater permanent deformations. In any event, as a result of the portion 17 (FIG. 6), virtually no supporting point at risk of perforation (23 in FIG. 5) occurs any longer in the probe tube 11.

If, however, the deflection 22 becomes so severe that the insertion aid 15 can no longer pass this buckling point, for instance because of the reduced remaining diameter of the interior tube space 25, the support of the insertion aid 15 against the buckling point has the effect in the interior of the tube 11 that, when pressure is exerted longitudinally on the proximal guide wire 13 according to FIG. 6, initially there is a buckling of the portion 17 in the probe tube 11 and then, because it is butting against the inner wall 24 of the tube 11, also deflection of the latter. This has the consequence that force components are transferred over the surface area radially onto the wall of the tube 11 and dissipated thereover, so that the remaining other, locally limited force component is usually no longer enough for the tube 11 to be punctured by the insertion aid 15 blocked at the buckling point.

What has been described above for the example of a nasogastric probe 11 can also be realized within the scope of the present invention in the case of intravascular insertion aids.

A guide wire 13 provided for use as a reinforcement in a pliable probe tube 11, while having radial play, therefore has proximally behind an insertion aid 15 first a flexible tip in the form of a more flexible portion 17 in comparison with the rest of the guide wire 13, before it is in turn adjoined proximally by the rest of this guide wire 13 of a conventional form. According to the invention, that portion 17 may be designed in such a way that, for this, the insertion aid 15 and the conventional rest of the guide wire 13 engage coaxially in ends opposite one another of an annular tube spring wound with or without prestress, in particular a flat-wire helical spring 19a of lower flexural stiffness wound under prestress on a block.

### LIST OF DESIGNATIONS

11 Tube (of 12)
12 Probe
13 Guide wire (with 17, 15; in 11)
14 Portion of wire (with 21; in 19/19a)
15 Insertion aid (at 16)
16 End (of 13, 17)
17 Portion (of reduced flexural stiffness; 13 proximally behind 15)
18 Solid wire (as 13, 17)
19 helical spring (as 17); 19a flat wire helical spring (as 17)
20 Outer lateral surface (of 13)
21 Rounding (as 15)
22 Deflection (of 11)
23 Supporting point (of 24 against 15 at 22)
24 Inner wall (of 11)
25 Interior space (of 11)

1-6. (canceled)

7. A guide wire (13) designed for inserting into the tube (11) of a nutritional probe (12) and provided at its end (16) with an insertion aid (15), wherein proximally behind the insertion aid (15) there is provided a portion (17) of the guide wire (13) of which the flexural stiffness is made to be less than that of the rest of the guide wire (13) proximally adjoining it, wherein a pliable portion (17) typically about 5 cm long of the guide wire (13) between the insertion aid (15) and the rest of the guide wire (13) is formed by a helical spring (19a) wound from flat wire under prestress on block, at which distally the rounded insertion aid (15) is formed and proximally the rest of the guide wire (13), this a stranded wire, engages into the spring (19a), in which it is fixed in a material-bonding manner.
